(19) United States
(12) Patent Application Publication
(10) Pub. No.: US 2025/0049421 A1
(43) Pub. Date: Feb. 13, 2025

(54) Ingestible Capsule with Beads for Sampling Content of the Gastrointestinal Tract

(71) Applicant: TRUSTEES OF TUFTS COLLEGE, Medford, MA (US)

(72) Inventors: Sameer Sonkusale, Lincoln, MA (US); Ruben Del Rio Ruiz, Candia, NH (US)

(21) Appl. No.: 18/834,377

(22) PCT Filed: Feb. 7, 2023
(86) PCT No.: PCT/US2023/012459

§ 371 Date: Jul. 30, 2024

Related U.S. Application Data
(60) Provisional application No. 63/307,896, filed on Feb. 8, 2022.

(51) Int. Cl. A61B 10/02; A61B 10/00

(52) U.S. Cl. CPC A61B 10/02; A61B 2010/0061

(57) ABSTRACT

An ingestible capsule for sampling liquid in the gastrointestinal tract includes an inlet and a valve. The valve includes an actuator bead that absorbs liquid. As it does so, it swells. This swelling closes the valve, thus preventing further liquid from entering the capsule.

### RELATED APPLICATIONS

This application claims the benefit of the Feb. 8, 2022 priority date of U.S. Provisional Application 63/307,896, the contents of which are incorporated herein by reference.

### STATEMENT OF GOVERNMENT RIGHTS

This invention was made with government support under grant AI144521 awarded by the National Institute of Health. The government has certain rights in the invention.

### FIELD OF INVENTION

The invention relates to sampling the content of the gastrointestinal tract and, in particular, to sampling using an ingestible sampler.

### BACKGROUND

A human being comprises a gastrointestinal tract that comprises a stomach, a large intestine, and a small intestine therebetween. As the food proceeds along the gastrointestinal tract, it undergoes various processes that result in extraction of nutrients. These nutrients are then absorbed into the human being's interior volume and used for various purposes.

Many portions of the gastrointestinal tract are hospitable to various microorganisms. This results in a “microbiome,” the composition of which changes as one traverses the gastrointestinal tract.

For example, the highly acidic stomach is somewhat of a microbial desert, with perhaps between ten and a thousand colony forming units per milliliter, most of which are lactobacillus, streptococcus, staphylococcus, and Enterobacteriaceae. As one crosses into the small intestine, the environment becomes more favorable, with the number of colony forming units rising to between 10,000 and 10,000,000 colony-forming units per milliliter. One also begins to encounter Bifidobacterium and Bacteroides. Meanwhile, the staphylococcus population dwindles.

Finally, one arrives at the large intestine. With its essentially neutral pH, the large intestine is truly teeming with life, with perhaps 10¹⁰ to 10¹¹ colony-forming units per milliliter of intestinal fluid. Moreover, the diversity of bacteria increases greatly, with Eubacterium, Clostridium, Pepto streptococcus, Fusobacteria now being present in many cases.

The composition of this microbiome depends, to some extent, on the health of the individual. In particular, certain gastrointestinal disorders are characterized in part by the presence and population of certain species. For example, alterations in the microbiome have been linked to such disorders as inflammatory bowel disease, irritable bowel syndrome, Crohn's disease, ulcerative colitis, and auto-immune disorders.

It is therefore useful to sample the microbiome in an effort to assess an individual's health.

A known way of learning about what bacteria is present in an individual's microbiome is to inspect that individual's fecal matter. While this method is useful for determining that a particular species exist, it is not so useful for sampling in a particular portion of the gastrointestinal tract.

A sample of fecal matter is, in effect, analogous to a path integral. Although it accumulates contributions from all points along the path (i.e., along the colon), it also discards location information in the process.

By way of analogy, it would be as if one wished to sample life on Earth by dragging a single bucket from the North Pole to the Equator. Based on the resulting sample, one would have no way to tell whether, for example, polar bears and monkeys share a common habitat.

Accordingly, the use of fecal matter as a bacterial sampling tool makes it difficult to understand how bacteria interact with the host and with each other.

There do exist methods for sampling the gut with somewhat greater spatial resolution. However, these tend to be invasive procedures that involve such activities as surgery, biopsy, and endoscopy. Among these procedures are those that also require considerable pre-operative cooperation from the patient, such as colonoscopy.

### SUMMARY

The invention concerns a capsule that comprises hydrophilic beads inside the capsule. The capsule has openings that allow gut fluids to enter. As the fluid enters, the beads absorb it and swell up. In doing so, they eventually swell up enough to close off the openings in the capsule. This locking mechanism helps prevent leakage and contamination of the sample.

Once the capsule is recovered, the content of the beads can be analyzed. Microbes that settle on the surface of the beads provide information indicative of the microbiome. In some embodiments, the beads are also functionalized with sensing chemistry, such as chemo responsive dyes, antibodies, or aptamers for targeted sensing of analytes.

In one aspect, the invention features an apparatus for sampling liquid from a gastrointestinal tract. The apparatus includes an ingestible capsule having an inlet and a valve, the valve comprising a bead that closes the valve after having absorbed liquid that enters the opening.

In some embodiments, the valve blocks the inlet when the bead is swollen with liquid that has entered the opening. In others, the valve comprises plates and a spring that urges the plates together. When swollen, the bead urges the plates apart.

Other embodiments include a channel having a first end at the inlet and a second end that opens into the capsule.

Still other embodiments include an additional bead that absorbs liquid but is outside the valve such that its swelling does not urge the valve to close.

Also among the embodiments are those in which the capsule is coated with a coating that prevents entry of liquid through the inlets until the coating has been lost during passage through the gastrointestinal tract.

A particularly suitable material for the bead is a superabsorbent polymer, such as sodium polyacrylate.

Embodiments further include those having a microfluidic diode that extends from the inlet into the capsule's interior. Such a microfluidic diode is oriented to promote flow into the capsule and inhibit flow out of the capsule.

Still other embodiments include those in which the bead has been functionalized to promote capture of bacteria by a surface of the bead.

Also among the embodiments are those in which the bead is one of many beads. These beads are between plates of the valve. During swelling thereof, the beads cooperate to urge the plates apart so as to close the valve.

Still other embodiments include those having a variable-weight ballast disposed in the capsule. The variable-weight ballast is configured to increase in weight during sampling of the liquid.

Also among the embodiments are those that include a fusible thread and a switch. The fusible thread is configured to urge the valve to remain closed. The switch, when closed, connects a voltage source to the fusible thread. The resulting electric current generates enough heat to destroy the fusible thread. Among these are embodiments in which the switch is a reed switch, in which case exposure of the reed switch to a magnetic field provided by the magnet opens the valve, thus initiating sampling of the liquid. Also among these are embodiments that comprise an electrochemical sensor that provides information on the capsule's environment. Based on such information, it is possible to infer that the capsule has arrived at a location at which sampling is to begin. In such cases, a switch, such as a transistor switch, is closed, thus causing current to generate heat that ultimately destroys the fusible thread.

Depending on how the electrochemical sensor has been functionalized, the measurement includes one or more of a measurement of acidity or alkalinity of the gastrointestinal tract, a measurement of one or more bile acids in the gastrointestinal tract, a measurement of glucose levels in the gastrointestinal tract, a measurement of lactate levels in the gastrointestinal tract, a measurement of dopamine levels in the gastrointestinal tract, a measurement of serotonin levels in the gastrointestinal tract, a measurement of amounts of short-chain fatty acids in the gastrointestinal tract, a measurement of oxygen in the gastrointestinal tract, and a measurement of a neurotransmitter in the gastrointestinal tract.

In some embodiments, the inlet defines an ellipse having a major axis that extends parallel to a longitudinal axis of the capsule.

In others, the bead changes appearance in response to exposure to an analyte.

In still other embodiments, the capsule comprises a nichrome wire that is disposed such that heating the nichrome wire results in commencement of sampling of the liquid.

In another aspect, the invention features a method that includes sampling liquid from a specified portion of a gastrointestinal tract. The method includes causing the liquid to begin entering the capsule when the capsule has arrived at the portion. The liquid that enters the capsule causes a bead in the capsule to swell with the liquid. This closes a valve in the capsule and prevents further entry of liquid. The method also includes recovering the capsule after the capsule has been ejected from the gastrointestinal tract.

Some practices include causing the liquid to begin entering the capsule by first coating the capsule with a coating. The coating is selected to be made from a material that is lost when the capsule arrives at the portion of the gastrointestinal tract.

In yet another aspect, the invention features an ingestible capsule for sampling fluid in the alimentary canal. Such a capsule features openings for admitting fluid into the capsule's interior and hydrophilic beads inside the capsule. The beads are configured to swell as liquid enters the openings up to a point at which the beads, after having been swollen, block the openings.

Embodiments include those with large elliptical inlets having inner valves and those with two-sided micro-channel valves.

In the embodiment having the elliptical inlets, an enteric coating on the capsule dissolves, thus leaving the inlets open.

Fluid that enters contacts the beads, which then start to expand. Once the beads are fully expanded, they push the internal valves outwards, blocking the two elliptical holes and thereby preventing further entry of fluid.

In the second embodiment, the beads push internal plates outward, thus blocking the inlets.

Embodiments further include those in which the beads comprise a super-absorbent polymer, such as sodium polyacrylate. Also among the embodiments are those in which the beads comprise hydrogels based on alginate, agarose, gelatin, PVP, CMC, Chitosan, polyethylene glycol, HEMA, pHEMA, TEGDMA, and PNIPAAM.

In alternative embodiments, the beads are functionalized on surfaces thereof to promote adhesion of microbes onto the surfaces.

A suitable material for making the capsule is an elastic and biocompatible resin such as 50 A resin, polytetrafluoroethylene, polypropylene, or polycarbonate. The essential requirement is that the capsule's casing be inert and that it does not leak sample through its walls.

Still other practices feature causing a fusible thread on the capsule to melt. This stops the fusible thread from holding the valve closed so that it now opens, only to be closed again later by the swelling of the beads. In some practices, this is carried out by holding a magnet to actuate a reed switch within the capsule. The reed switch then closes and allows current to heat up a wire, which then causes the fusible thread to melt. In other practices, this is carried out by using information from an electrochemical sensor on the capsule trigger as a basis for closing a switch, such as a transistor switch, to connect a voltage source to the thread, which then heats up in response to current flowing therethrough, thus melting the capsule. In such practices, the switch is closed when the capsule's environment, as sensed by the electrochemical sensor, has reached a pre-defined state at which sampling should begin.

Among the advantages of the capsule are the ability to conduct site-directed drug delivery, real-time imaging and sensing of gut biomarkers, and diagnosis and treatment of certain gastrointestinal conditions.

In some embodiments, the capsule contains both the gastric fluid, including its metabolites, cytokines, and chemokines.

In some embodiments, the beads sample small molecules that enter along with the water during the swelling process.

Embodiments also include those in which microbes stay on the surfaces of the beads and those in which they enter the beads. The latter is implemented by using hydrogel beads.

Upon recovery, the pill is cut open and microbes, which are mostly trapped on the beads' surfaces, are analyzed. In addition, collected fungus and virus particles, if any, are analyzed.

The beads are then analyzed for chemical composition of molecules trapped therein, for example using a standard assay such as a sandwich assay, an immunoassay, LC-MS, Raman, or NMR.

Embodiments further include those in which the beads serve as sensors. Examples include those in which the beads fluoresce or change in color in the presence of one or more analytes.

In still other embodiments, beads are functionalized directly to detect a certain bacteria or virus or fungi without the need to do complicated assay. This can be carried out, for example, by functionalizing with antibodies, nanobodies, enzymes or aptamers that bind to the target analyte. Among these are embodiments where the binding agent changes color or engages in fluorescence. This enables optical readout. Also among these are embodiments suitable for electrochemical readout, such as those in which the binding agent causes a change in impedance or current levels when plugged in between electrodes.

### BRIEF DESCRIPTION OF THE FIGURES

These and other features of the invention will be apparent from the following detailed description and the accompanying figures, in which:

FIG. 1 shows an ingestible capsule having a valve in the open position to permit entry of fluid therein;

FIG. 2 shows the capsule of FIG. 1 with the valve having been closed as a result of beads having become swollen;

FIG. 3 shows an alternative capsule having microchannels to inhibit flow out of said capsule;

FIG. 4 shows a capsule having a fusible thread that, when melted, initiates the sampling process;

FIG. 5 shows an embodiment similar to that in FIG. 5 but that relies on a sensor to establish when sampling should begin;

FIG. 6 shows an example of circuitry used for the embodiment shown in FIG. 5; and

FIG. 7 shows details of a shut-down circuit from FIG. 6.

### DETAILED DESCRIPTION

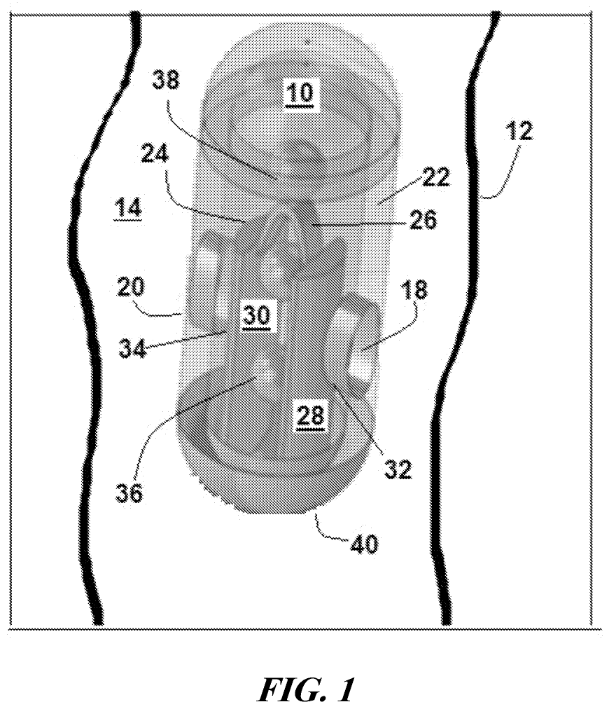

FIG. 1 shows an ingestible capsule 10 that, soon after being swallowed, traverses an intestine 12 filled with intestinal fluid 14 that is to be sampled. Suitable materials for use in making a capsule 10 are those that are inert and discourage leakage through its walls. Examples include inert plastics, such as such as polytetrafluoroethylene, polypropylene, and polycarbonate.

The capsule 10 has a wall 16 having first and second inlets 18, 20 through which intestinal fluid 14 enters a sample chamber 22 within the capsule 10. In the illustrated embodiment, the inlets 18, 20 are elliptical with a major axis parallel to that along which the capsule 10 extends.

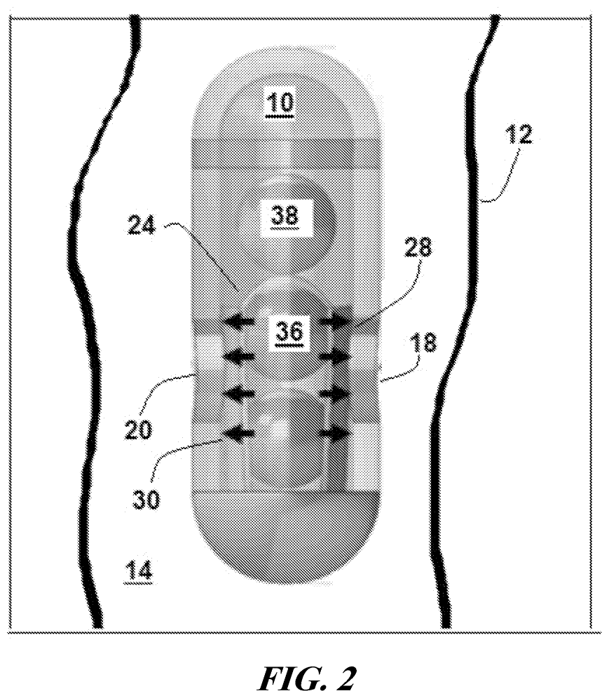

The capsule 10 also includes a valve 24. FIG. 1 shows the valve 24 in its open position. In FIG. 2, the valve 24 is in its closed position.

The valve 24 comprises a spring 26 that couples to corresponding ends of first and second plates 28, 30. The spring 26 exerts a force that biases the plates 28, 30 so that they resist being pushed apart. At equilibrium, the valve 24 is thus in the open position shown in FIG. 1.

The first plate 28 faces the first inlet 18 across a first gap 32. The second plate 30 faces the second inlet 20 across a second gap 34. With the valve 24 in the open position, intestinal fluid 14 enters the capsule 10 through the first and second inlets 18, 20.

The valve 24 also includes actuator beads 36. The actuator beads 36 are nestled between the plates 28, 30.

When dry, the actuator beads 36 have a diameter that just fits the available space between the plates 28. 30. As the capsule 10 fills with intestinal fluid 14, the actuator beads 36 begin to swell. Eventually, the actuator beads 36 swell sufficiently to overcome the bias force of the spring 26. The plates 28, 30 thus move slowly apart until they eventually come to rest on the inlets 18, 20. At this point, the valve 24 will have transitioned into its closed state, which can be seen in FIG. 2.

It has been observed that the actuator beads 36 carry out a function other than swelling in response to exposure to liquid. The surface of the actuator beads 36 is such that microorganisms have a pronounced tendency to preferentially adhere to their surfaces.

For this reason, it is advantageous to provide one or more collection beads 38 that are identical to the actuator beads 36. Unlike the actuator beads 36, the collection beads 38 are not between the plates 28, 30. As such, they do not urge the valve 24 closed as they swell, as do the actuator beads 36.

Nevertheless, collection beads 38 do participate in concentrating microorganisms. They do so because microorganisms preferentially adhere to the surfaces of the collection beads 38 for the same reason that they adhere to the surfaces of the actuator beads 36.

The beads 36, 38 are made of a highly absorbent material. In some embodiments, the beads 36, 38 comprise a superabsorbent polymer, such as sodium polyacrylate. Other embodiments include beads 36, 38 comprising a hydrogel based on one or more of alginate, alginate, agarose, gelatin, PVP, CMC, Chitosan, polyethylene glycol, HEMA, pHEMA, TEGDMA, and PNIPAAM. In other embodiments, the beads 36, 38 have been functionalized to promote adhesion of bacteria thereto.

Since the valve 24 remains open at equilibrium, a capsule 10 as described thus far would fill up with fluid before it got very far along the gastrointestinal tract 12. Accordingly, it would be useless for sampling the microbiome in the large intestine.

The capsule 10 is coated with a coating 40 that disintegrates when the environment surrounding the capsule 10 attains a particular condition. This coating 40 would thus seal the inlets 18, 20 until the capsule 10 is within that portion of the gut that is to be sampled, at which point the coating 40 dissolves so that intestinal fluid 14 can enter the capsule 10. As the intestinal fluid 14 enters the capsule 10, the beads 36, 38 swell and cause the inlets 18, 20 to be blocked by the plates 28, 30. This avoids further exchange of intestinal fluid 14 between the capsule 10 and its environment, thus avoiding contamination of the sample within the sample chamber 22.

A particularly useful embodiment is one in which the integrity of the coating 40 depends on acidity. In such a case, the coating 40 would cover the inlets 14, 16 while the capsule 10 traverses a highly acidic environment, such as that of the stomach. Then, upon reaching the more neutral environment of the colon 12, the coating 40 would dissolve, thereby exposing the inlets 14, 16 and permitting entry of intestinal fluid 14.

A disadvantage of the capsule 10 shown in FIGS. 1 and 2 is that fluid entering the capsule 10 can easily exit the capsule 10. As a result, the composition of the intestinal fluid 14 within the capsule 10 at the end of the capsule's journey through the intestine 12 may not reflect the composition of the fluid at the region of the intestine 12 that was intended to be sampled.

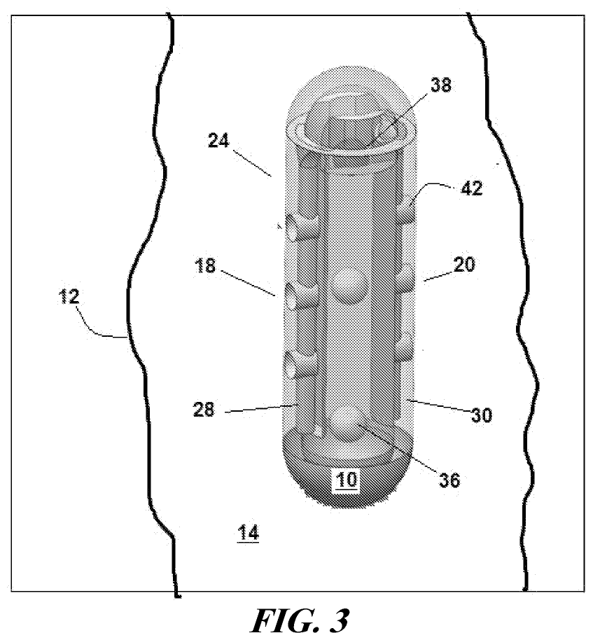

FIG. 3 shows an alternative embodiment to reduce the likelihood of this contaminating a sample from one region with species from another region, an alternative embodiment. The embodiment shown in FIG. 3 takes advantage of the asymmetry in the driving force for flow. Fluid that enters the capsule 10 has the benefit of being driven by peristaltic action of the gastrointestinal tract 12. In contrast, fluid that leaves the capsule 10 depends on diffusion alone.

To harness this asymmetry, the capsule 10 provides long narrow channels 42 leading inward from the two inlets 18, 20. As a result of their geometry, these channels 40 offer high flow resistance that tends to impede flow out of the capsule 10 far more than flow into the capsule 10.

The channels 40 function in a manner analogous to diodes. As a result, the composition of the fluid within the capsule is more likely to reflect that which existed at the time that the inlets 14, 16 were first opened to admit fluid into the capsule 10.

In the embodiments described this far, the capsule 10 is ingested with its valve 24 open. This means that, without the coating 40, the capsule 10 would quickly fill with liquid. The coating 40 delays the onset of the sampling process until the environment of the gastrointestinal tract 12 has the properties required to dissolve or disintegrate the coating 40.

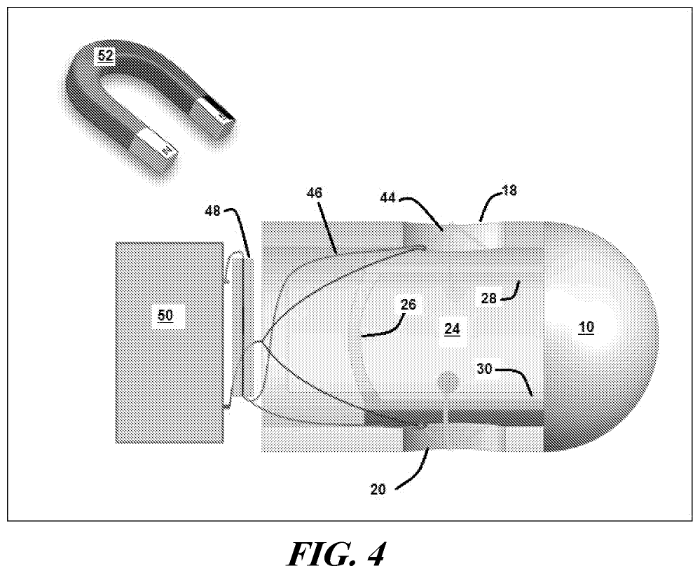

FIG. 4 shows an alternative embodiment of the capsule in which the onset of sampling relies on loss of a component other than the coating 40. A portion of the capsule's wall has been omitted for clarity. In addition, the beads 36, 38 have also been omitted for clarity.

Like the valve 24 shown in FIGS. 1 and 2, the illustrated capsule's valve 24 has a spring 26 that urges the plates 28. 30 inward. In addition, the valve 24 includes a fusible thread 44 that anchors the plates 28, 30 outward so that the spring 26 cannot pull the plates 28, 30 inward. As a result, the plates 28, 30 close the valve 24. As a result, the capsule 10 is ingested with the valve 24 closed.

A wire 46 and a switch 48 connect the fusible thread 44 to a voltage source 50. The switch 48 is a reed switch that is actuatable from outside the body by a magnet 52. A suitable wire 46 is one that generates considerable heat in response to electric current. An example of a suitable wire 46 is one made of nichrome.

When it is time to begin sampling, a clinician holds the magnet 52 over the patient's abdomen at the approximate location of the capsule 10. This closes the switch 48, thus causing considerable current to flow down the wire 46. The resulting ohmic heating melts the fusible thread 46. As a result, the spring 26 is free to open the valve 24. As was described in connection with FIGS. 1 and 2, the beads 36, 38 eventually swell enough to close the valve 24, thus completing the sampling process.

A disadvantage of the embodiment shown in FIG. 4 is the need for human intervention. A clinician must somehow infer the location of the capsule 10 and stand ready with a magnet to actuate a reed switch within the capsule 10.

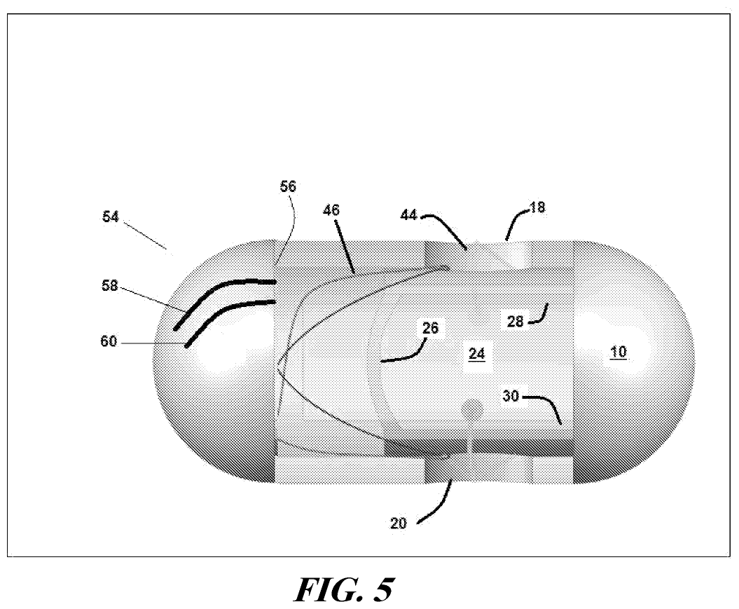

An alternative embodiment, which is shown in FIG. 5, avoids reliance on human intervention. The embodiment shown in FIG. 5 instead relies on an electrochemical sensor 54 that monitors the capsule's surrounding environment. Based on the properties of the environment, it is possible to infer the location of the capsule 10 in the gastrointestinal tract, and hence whether or not sampling should begin. A capsule's arrival at a location with predefined environmental properties is thus used to close a switch, which then ultimately destroys the fusible thread 46 so that sampling can begin.

In the embodiment shown in FIG. 5, the electrochemical sensor 54 comprises first and second threads 58, 60 nestled within the recesses on the capsule's surface. The first and second threads 58, 60 connect to the circuitry 56 via corresponding openings at one end of the recesses.

In some embodiments, the electrochemical sensor 54 comprises conductive material that has been printed directly on the capsule's outer surface during an additive manufacturing process. In such embodiments, recesses are optional. The conductive material that has been printed onto the outer surface connects to the circuitry 56 through corresponding vias. In such embodiments, the capsule's wall is essentially a printed-circuit board that exists on a curved manifold.

In those embodiments that are configured to sense acidity or alkalinity the first thread 58 is a reference electrode that comprises silver and the second thread 60 is a working electrode that comprises a carbon-coated linen thread that has been further coated with a conducting polymer, such as polyaniline.

Within the gastrointestinal tract 12, there are many other parameters other than acidity or alkalinity that may be of interest.

For example, in some embodiments, it is useful to sense the concentration of dissolved oxygen. In addition, it is useful to be able to measure concentrations of such substances that are known to be high at selected locations since doing so provides an indication of where the capsule 10 is located. For example, an electrochemical sensor 54 that is tuned to sense bile would provide a way to recognize that the capsule 10 has reached the neighborhood at which the bile duct empties into the intestine.

The electrochemical sensor 54 is by no means limited to sensing only one parameter. There is plenty of space on capsule's outer surface for threads 58, 60 or conductive strips that have been functionalized in different ways to sense different substances or environmental conditions within the gastrointestinal tract 12.

In effect, the set of all measurable properties at a location within the gastrointestinal tract 12 defines a multi-dimensional space. Measurement of one or more coordinates of a point in that space provides information from which it is possible to make inferences concerning the location of the capsule at the time of measurement. The more coordinates one samples using the electrochemical sensor 54, the more precisely one can distinguish the location of the capsule 10. For example, if two locations have the same pH, one could not distinguish them by measuring only pH. But if one location differed from the other in dissolved oxygen concentration, then a measurement of those two coordinates in the measurement space would provide a basis for distinguishing the two locations.

The use of pH and pO2 (i.e., partial pressure of dissolved oxygen) rather than pH alone is particularly helpful because pH varies only within a narrow range in both the small and large intestines, because it fluctuates locally, and because the spatial distribution of pH is likely to vary among individuals. The use of pH and pO2 in concert provides enough spatial resolution to determine whether the capsule 10 is within the stomach, duodenum, jejunum, ileum, ascending colon or descending colon.

Embodiments of the electrochemical sensor 54 thus include sensors that have been configured to measure any one or more of: acidity, alkalinity, presence of bile, glucose levels, lactate levels, dopamine levels, serotonin levels, oxygen levels, and concentrations of various fatty acids, such as short-chain fatty acids.

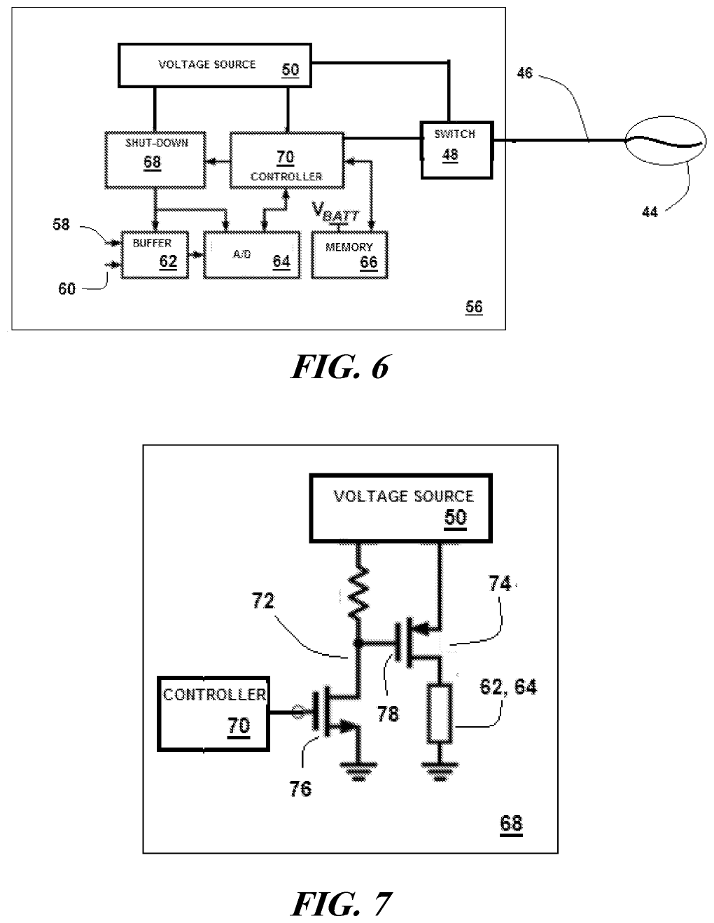

Referring now to FIG. 6, the electrochemical sensor 54 interfaces with a voltage buffer 62 that provides an analog signal to an analog-to-digital converter 64. The analog-to-digital converter 64 converts the analog signal from the electrochemical sensor 54 into data that can be stored in a memory 66, such as an EEPROM. In a preferred embodiment, the analog-to-digital converter 64 is a delta-sigma converter, and in particular, a 16-bit ΔΣ converter.

Operation of the electrochemical sensor 54 consumes considerable amounts of energy. In an effort to conserve the finite supply of energy provided by the voltage source 50, particularly given that the voltage source 50 must have enough available to melt the fusible thread 44, it is useful to observe the environment only when the capsule 10 is traversing a region of interest within the tract. It is thus useful to provide a shut-down circuit 68. This shut-down circuit 68 enables or disables operation of the analog-to-digital converter 64 and the voltage buffer 62 in response to commands from a microcontroller 70.

In response to a signal from the microcontroller 70, the shutdown circuit 68 permits the voltage buffer 62 to buffer an analog signal from the electrochemical sensor 54. It also permits the analog-to-digital converter 64 to convert that analog signal into data that it then provides the microcontroller 70.

The microcontroller 70 receives this data and determines whether or not the environmental conditions are such that sampling of the intestinal fluid 14 should begin. If it determines that the capsule 10 has arrived at a location suitable for sampling, the microcontroller 70 sends a signal to connect the voltage source 50 the fusible thread 44, thereby melting it and allowing sampling to commence, as described in connection with FIG. 4.

FIG. 7 shows a particular implementation of a shutdown circuit 68 featuring first and second conducting paths 72, 74, both of which connect the voltage source 50 to ground. The load, i.e., the voltage buffer 62 and the analog-to-digital converter 64, lies along the second conducting path 74.

First and second transistors 76, 78 that lie along the first and second conducting paths 72, 74 cause the paths 72, 74 to transition between conducting and non-conducting states. The microcontroller 70 provides a signal to the first transistor's gate. Meanwhile, the second transistor's gate connects to the first conducting path 72 at a point whose voltage depends on whether the first transistor 76 is allowing the first conducting path 72 to conduct. As a result, the microcontroller 70 also controls flow along the second conducting path 74, thereby enabling or disabling the voltage buffer 62 and the analog-to-digital converter 64.

A suitable material for use in a fusible thread 44 is fusible nylon having a mass density of approximately 167 dtex and a melt temperature of 60° C. A suitable wire 46 is a nine-millimeter long 36G wire made of 40 nichrome alloy. A suitable voltage source 50 is one that begins at 2.7 volts and provides about 0.4 amperes to fuse the nylon.

In some embodiments, the capsule 10 comprises a ballast 38 that maintains stability during the sampling of the liquid. Preferably, the ballast 38 is a variable-weight ballast that increases its weight during the sampling process. Such a ballast is achieved by using a mass of a hydrophilic material, such as a super-absorbent polymer.

Having described the invention and a preferred embodiment thereof. what is claimed as new and secured by letters patent is:

1. An apparatus for sampling liquid from a gastrointestinal tract, said apparatus comprising an ingestible capsule comprising an inlet, a valve, and a bead that closes said valve after having absorbed liquid that enters said opening.

2. The apparatus of claim 1, wherein said valve blocks said inlet when said bead is swollen with liquid that has entered said opening.

3. The apparatus of claim 1, wherein said valve comprises plates and a spring that urges said plates together, wherein said bead, when swollen, urges said plates apart.

4. (canceled)

5. The apparatus of claim 1, wherein said bead is a first bead and wherein said apparatus further comprises a second bead that absorbs said liquid, wherein said second bead is outside said valve such that swelling of said second bead does not urge said valve to close.

6. (canceled)

7. (canceled)

8. The apparatus of claim 1, further comprising a microfluidic diode that extends from said inlet into an interior of said capsule, said microfluidic diode being oriented to promote flow into said capsule and inhibit flow out of said capsule.

9. (canceled)

10. The apparatus of claim 1, wherein said bead is one of a plurality of beads that are between plates of said valve, wherein, during swelling thereof, said beads cooperate to urge said plates apart so as to close said valve.

11. The apparatus of claim 1, further comprising a variable-weight ballast disposed in said capsule, wherein said variable-weight ballast is configured to increase in weight during sampling of said liquid.

12. The apparatus of claim 1, further comprising a fusible thread that is configured to urge said valve to remain closed and a switch that, when closed, connects a voltage source to said fusible thread, thereby destroying said fusible thread.

13. The apparatus of claim 1, further comprising a magnet and a reed switch, wherein exposure of said reed switch to a magnetic field provided by said magnet causes said valve to open, thereby initiating sampling of said liquid.

14-28. (canceled)

29. The apparatus of claim 1, further comprising an electrochemical sensor, a controller, a switch, a wire, and a fusible thread, wherein said wire connects said fusible thread to said voltage source via said switch, wherein said electrochemical sensor is disposed on said capsule to monitor an environment of said capsule and to provide information representative of said environment, and wherein said controller controls said switch based at least in part on said information.

30. The apparatus of claim 29, wherein said electrochemical sensor is functionalized for measurement of acidity or alkalinity of said gastrointestinal tract.

31. The apparatus of claim 29, wherein said electrochemical sensor is functionalized for measurement of bile in said gastrointestinal tract.

32. The apparatus of claim 29, wherein said measurement is a measurement of glucose levels in said gastrointestinal tract.

33. The apparatus of claim 29, wherein said electrochemical sensor is functionalized for measurement of lactate levels in said gastrointestinal tract.

34. The apparatus of claim 29, wherein said measurement is a measurement of dopamine levels in said gastrointestinal tract.

35. The apparatus of claim 29, wherein said electrochemical sensor is functionalized for measurement of serotonin levels in said gastrointestinal tract.

36. The apparatus of claim 29, wherein said electrochemical sensor is functionalized for measurement of amounts of short-chain fatty acids in said gastrointestinal tract.

37. The apparatus of claim 29, wherein said electrochemical sensor is functionalized for measurement of dissolved oxygen concentration in said gastrointestinal tract.

38. The apparatus of claim 29, wherein said wherein said electrochemical sensor is functionalized for measurement of a neurotransmitter in said gastrointestinal tract.

39. A method comprising sampling liquid from a specified portion of a gastrointestinal tract, said method comprising causing said liquid to begin entering said capsule when said capsule has arrived at said portion, wherein liquid that enters said capsule causes a bead in said capsule to swell with said liquid, thereby closing a valve in said capsule and preventing further entry of liquid, said method further comprising recovering said capsule after said capsule has been ejected from said gastrointestinal tract.

40-41. (canceled)
